(19) United States
(12) Patent Application Publication
(10) Pub. No.: US 2024/0173498 A1
(43) Pub. Date: May 30, 2024

(54) CPAP MACHINE

(71) Applicant: Murata Manufacturing Co., Ltd., Kyoto (JP)

(72) Inventors: Yoshio YAMANAKA, Kyoto (JP); Masayuki SHIRAI, Kyoto (JP); Shin HASEGAWA, Kyoto (JP); Kenghua HSING, Kyoto (JP)

(21) Appl. No.: 18/497,245

(22) Filed: Oct. 30, 2023

(30) Foreign Application Priority Data
Nov. 30, 2022 (JP) 2022-191515

(51) Int. Cl. A61M 16/00; A61M 16/08; A61M 16/16

(52) U.S. Cl. CPC A61M 16/024; A61M 16/0066; A61M 16/0816; A61M 16/0875; A61M 16/161; A61M 2205/3368

(57) ABSTRACT

A CPAP machine includes a housing, an air pipe, a blower, and a sensor S. The housing includes a first opening and a second opening. The air pipe is disposed in the housing, and has an inlet port continuous with the first opening, and an outlet port. The sensor S measures a temperature or a humidity of a gas that flows through an internal space of the air pipe. The air pipe includes a box that protrudes outward from a wall of the air pipe and a wall disposed in the box. The wall is disposed in the box to define a space continuous with the internal space of the air pipe, a space, and a space that connects the space and the space to each other. The sensor S is disposed at a wall of the box.

### CROSS REFERENCE TO RELATED APPLICATION

This application claims priority from Japanese Patent Application No. 2022-191515 filed on Nov. 30, 2022. The content of this application is incorporated herein by reference in its entirety.

### BACKGROUND OF THE DISCLOSURE

### 1. Field of the Disclosure

The present disclosure relates to a continuous positive airway pressure (CPAP) machine that supplies users with a gas at controlled temperature and humidity.

### 2. Description of the Related Art

Japanese Unexamined Patent Application Publication No. 2007-170762 describes a humidifier. The humidifier described in Japanese Unexamined Patent Application Publication No. 2007-170762 includes a humidity sensor on a printed-circuit board.

The printed-circuit board includes a protruding portion, to which a temperature sensor is attached. An airway of the humidifier extends to surround the protruding portion.

To supply users with a gas at controlled temperature and humidity, continuous positive airway pressure (CPAP) machines have been currently put to practical use. A gas delivered by a CPAP machine is directly supplied to the respiratory organs of a user through the mouth and nose. Thus, the gas is required to have its temperature and humidity fully controlled.

However, in the humidifier described in Japanese Unexamined Patent Application Publication No. 2007-170762, a small air current hits against the sensor that measures the temperature or humidity. Thus, the sensor may fail to be fully cooled, and cause a large error in measurement of the temperature or humidity. Thus, directly applying the mechanism of the humidifier described in Japanese Unexamined Patent Application Publication No. 2007-170762 to a CPAP machine would result in failure to sufficiently control the temperature and humidity.

### BRIEF SUMMARY OF THE DISCLOSURE

Accordingly, it is a possible benefit of the present disclosure to provide a CPAP machine having an improved effect of cooling a sensor.

The CPAP machine of the present disclosure includes a housing, an air pipe, a blower, and a sensor. The housing includes a first opening and a second opening. The air pipe is disposed in the housing, and has an inlet port continuous with the first opening, and an outlet port. The blower is disposed in the housing, and guides a gas that has flowed in from the outlet port to the second opening. The sensor measures a temperature or a humidity of a gas that passes through an internal space of the air pipe.

The air pipe includes a protrusion that protrudes outward from a wall of the air pipe, and a diverting wall disposed in the protrusion. The diverting wall is at least partially located in the protrusion. The diverting wall defines a first communicating port and a second communicating port together with a protrusion defining wall that defines the protrusion. The first communicating port and the second communicating port are disposed to be continuous with each other inside the protrusion. The sensor is disposed at the protrusion defining wall.

In this structure, part of air that flows through a main space of the air pipe returns to the main space through the interior of the protrusion. Thus, an air current parallel to a wall defining the protrusion occurs. This air current cools the sensor. Specifically, the CPAP machine diverts part of an air current used as a function of the CPAP machine to cool the sensor.

The present disclosure can improve the effect of cooling the sensor.

### BRIEF DESCRIPTION OF THE SEVERAL VIEWS OF THE DRAWINGS

FIG. 1 is an external perspective view of a CPAP machine according to an embodiment of the present disclosure;

FIG. 2A is a plan view of a CPAP machine according to an embodiment of the present disclosure,

FIG. 2B is a first side view of a CPAP machine according to an embodiment of the present disclosure, and

FIG. 2C is a second side view of a CPAP machine according to an embodiment of the present disclosure;

FIG. 3 is an external perspective view of an air pipe according to an embodiment of the present disclosure and a schematic view of an air current;

FIG. 4A is a plan view of an air pipe according to an embodiment of the present disclosure,

FIG. 4B is a first side view of an air pipe according to an embodiment of the present disclosure, and

FIG. 4C is a second side view of an air pipe according to an embodiment of the present disclosure;

FIG. 5A is an enlarged plan view of a portion of a box according to an embodiment of the present disclosure,

FIG. 5B is an enlarged first side view of a portion of a box according to an embodiment of the present disclosure, and

FIG. 5C is an enlarged second side view of a portion of a box according to an embodiment of the present disclosure;

FIG. 6 is a further enlarged side cross-sectional view of a portion of a box according to an embodiment of the present disclosure;

FIGS. 7A and 7B are enlarged cross-sectional views of other examples of an arrangement of a sensor according to an embodiment of the present disclosure;

FIGS. 8A and 8B are enlarged cross-sectional views of other examples of a diverting wall according to an embodiment of the present disclosure;

FIG. 9 is a view of another example of an arrangement of a box according to an embodiment of the present disclosure; and

FIG. 10 is a view of another example of a box according to an embodiment of the present disclosure.

### DETAILED DESCRIPTION OF THE DISCLOSURE

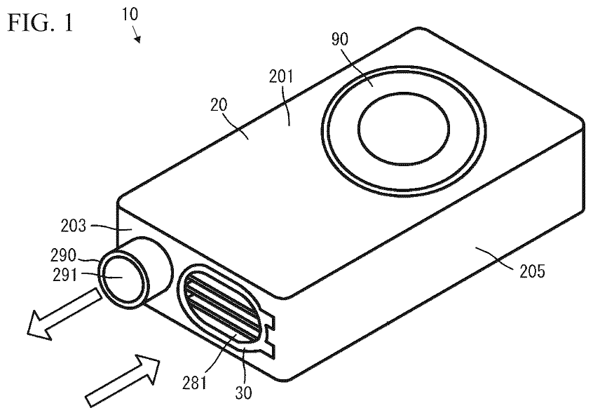

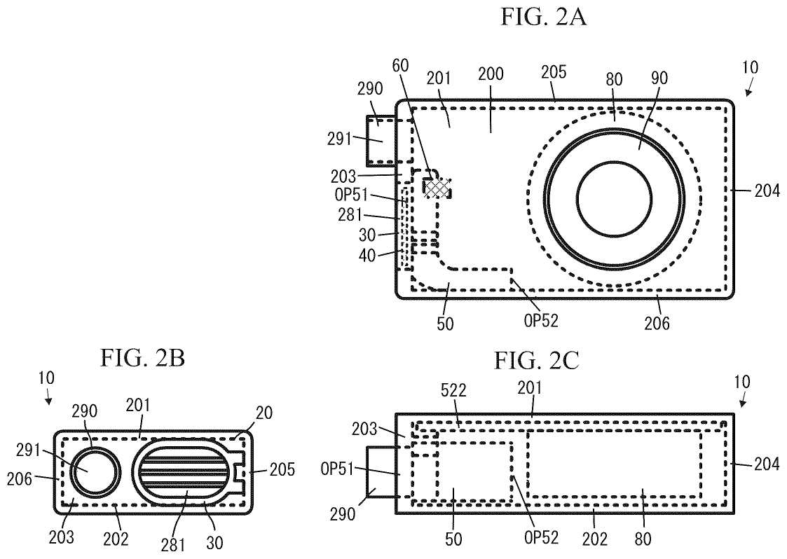

A CPAP machine according to an embodiment of the present disclosure is described with reference to the drawings. FIG. 1 is an external perspective view of a CPAP machine according to an embodiment of the present disclosure. FIG. 2A is a plan view of the CPAP machine according to the embodiment of the present disclosure. FIG. 2B is a first side view of the CPAP machine according to the embodiment of the present disclosure. FIG. 2C is a second side view of the CPAP machine according to the embodiment of the present disclosure. The plan or the side described in embodiments including the present embodiment are named merely for convenience of illustration, and not intended to limit the use of the CPAP machine.

As illustrated in FIG. 1, FIG. 2A, FIG. 2B, and FIG. 2C, a CPAP machine 10 includes a housing 20, an air pipe 50, a circuit board 522, a blower 80, and an operation button 90.

The housing 20 has a substantially rectangular parallelepiped shape, and includes an upper wall 201, a lower wall 202, a front wall 203, a rear wall 204, a side wall 205, and a side wall 206. The upper wall 201 and the lower wall 202 face each other. The front wall 203 and the rear wall 204 face each other. The side wall 205 and the side wall 206 face each other.

The front wall 203, the rear wall 204, the side wall 205, and the side wall 206 are connected to the peripheries of the upper wall 201 and the lower wall 202. Thus, the housing 20 includes a housing internal space 200 defined by the front wall 203, the rear wall 204, the side wall 205, and the side wall 206.

The front wall 203 has a first opening 281 and a second opening 291. The first opening 281 and the second opening 291 extend through the front wall 203 in a thickness direction. The first opening 281 and the second opening 291 connect the housing internal space 200 of the housing 20 to the external space of the housing 20. Functionally, the first opening 281 serves as an inlet port of the housing 20, and the second opening 291 serves as an outlet port of the housing 20.

An air exhaust path 290 is installed at the front wall 203. The air exhaust path 290 protrudes from an outer surface of the front wall 203. A center opening of the air exhaust path 290 is connected to the second opening 291 formed in the front wall 203, and this portion also functions as the second opening 291.

When the first opening 281 is viewed from the front, an air filter 40 is disposed at the housing 20 at a position to overlap the first opening 281.

When the first opening 281 is viewed from the front, a filter cover 30 is installed at the front wall 203 of the housing 20 at a position to overlap the first opening 281 on which the air filter 40 is disposed. The filter cover 30 includes multiple slits defined by multiple pillars, and enables ventilation with the multiple slits. The air filter 40 is held by the housing 20 as a result of part (protrusions of the multiple pillars and a frame) of the filter cover 30 being in contact with the air filter 40.

The air pipe 50, the circuit board 522, and the blower 80 are disposed inside (in the housing internal space 200 of) the housing 20.

The air pipe 50 is tubular, and has one opening end of the tube serving as an inlet port OP51, and the other opening end of the tube serving as an outlet port OP52. The inlet port OP51 of the air pipe 50 is connected to the first opening 281 of the housing 20. The outlet port OP52 of the air pipe 50 is disposed adjacent to the blower 80 in the housing internal space 200. A specific shape of the air pipe 50 is described below.

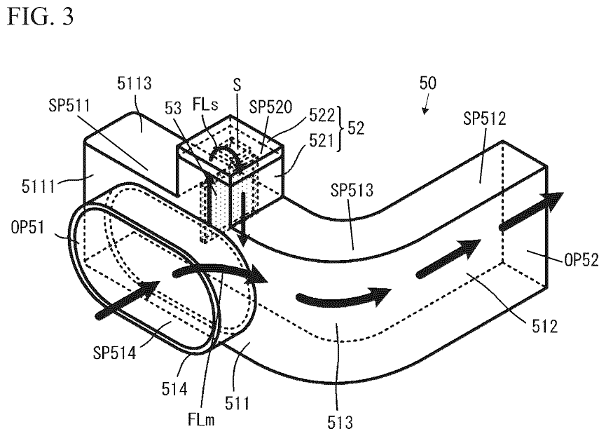

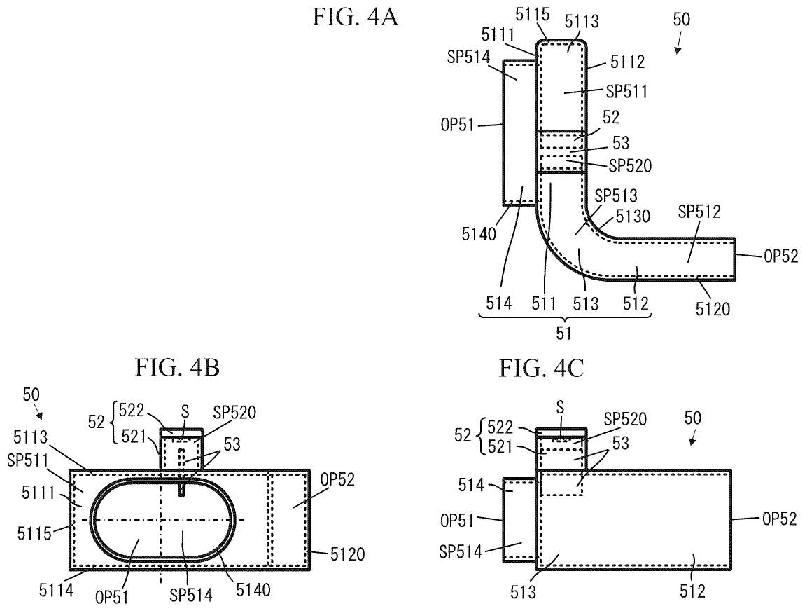

The circuit board 522 is disposed adjacent to the upper wall 201 in the housing internal space 200. A main flat surface of the circuit board 522 and a surface of the upper wall 201 facing the housing internal space 200 are substantially parallel to each other. A sensor S (refer to, for example, FIG. 3 and FIGS. 4A to 4C) is mounted on the circuit board 522. The circuit board 522 is electrically connected to a motor of the blower, and connected to the operation button 90. The circuit board 522 detects an operation on the operation button 90, and controls driving and stopping of the blower 80 using this detection result.

The blower 80 includes, for example, a motor and fins. The blower 80 blows air by rotating the fins, and controls the number of rotations of the fins to adjust the quantity of airflow.

When the blower 80 is driven, a gas is sucked into the housing 20 through the first opening 281. Since the first opening 281 and the inlet port OP51 are connected to each other, the gas sucked through the first opening 281 flows into the air pipe 50 from the inlet port OP51. The gas that has passed through the air pipe 50 flows into the housing internal space 200 from the outlet port OP52. The blower 80 guides this gas to the second opening 291, and discharges the gas out of the housing 20 from the second opening 291.

Thus, the CPAP machine 10 can supply the gas to the user at a desired quantity of flow (pressure).

### Air Pipe 50

FIG. 3 is an external perspective view of an air pipe according to an embodiment of the present disclosure and a schematic view of an air current. FIG. 4A is a plan view of an air pipe according to an embodiment of the present disclosure, FIG. 4B is a first side view of an air pipe according to an embodiment of the present disclosure, and FIG. 4C is a second side view of an air pipe according to an embodiment of the present disclosure.

As illustrated in FIG. 3, FIG. 4A, FIG. 4B, and FIG. 4C, the air pipe 50 includes a first tubular body 511, a second tubular body 512, a third tubular body 513, a fourth tubular body 514, a box 52, and a wall 53. The wall 53 corresponds to a diverting wall of the present disclosure.

The first tubular body 511 has a tube shape having a longitudinal direction. The first tubular body 511 includes a wall 5111, a wall 5112, a wall 5113, a wall 5114, and a wall 5115. The wall 5111, the wall 5112, the wall 5113, and the wall 5114 are parallel to the longitudinal direction of the first tubular body 511. The wall 5115 is perpendicular to the longitudinal direction of the first tubular body 511. The wall 5111 and the wall 5112 face each other. The wall 5113 and the wall 5114 face each other. The wall 5111 and the wall 5112 are connected to the wall 5113 and the wall 5114. The wall 5115 is connected to one ends of the wall 5111, the wall 5112, the wall 5113, and the wall 5114 in the longitudinal direction. Thus, the first tubular body 511 has a first internal space SP511.

The second tubular body 512 includes a tubular wall 5120 having a longitudinal direction. The longitudinal direction of the second tubular body 512 is substantially perpendicular to the longitudinal direction of the first tubular body 511. The second tubular body 512 has a second internal space SP512 enclosed by the wall 5120.

The third tubular body 513 includes a curved tubular wall 5130 in a plan view. The third tubular body 513 has a third internal space SP513 enclosed by the wall 5130. The third tubular body 513 is connected to the first tubular body 511 and the second tubular body 512. Thus, the third internal space SP513 is connected to the first internal space SP511 and the second internal space SP512.

The fourth tubular body 514 includes a tubular wall 5140, and has a fourth internal space SP514 enclosed by the wall 5140. The wall 5140 of the fourth tubular body 514 is connected to the wall 5111 of the first tubular body 511. The first tubular body 511 has an opening at a portion to which the fourth tubular body 514 is connected. Thus, the fourth internal space SP514 is connected to the first internal space SP511.

The air pipe 50 with the above components has a structure where the fourth tubular body 514, the first tubular body 511, the third tubular body 513, and the second tubular body 512 are connected in this order. The internal space of the air pipe 50 is connected to the outside of the air pipe 50 at the end closer to the fourth tubular body 514 and at the end closer to the second tubular body 512. The space including the first internal space SP511, the second internal space SP512, the third internal space SP513, and the fourth internal space SP514 corresponds to a main space of the air pipe of the present disclosure.

The opening in the air pipe 50 closer to the fourth tubular body 514 serves as the inlet port OP51 of the air pipe 50. The opening in the air pipe 50 closer to the second tubular body 512 serves as the outlet port OP52 of the air pipe 50. Specifically, a gas that has flowed into the housing 20 flows into the air pipe 50 through the inlet port OP51, passes through the fourth internal space SP514 of the fourth tubular body 514, the first internal space SP511 of the first tubular body 511, the third internal space SP513 of the third tubular body 513, and the second internal space SP512 of the second tubular body 512, and is discharged from the outlet port OP52.

### Box 52 and Wall 53

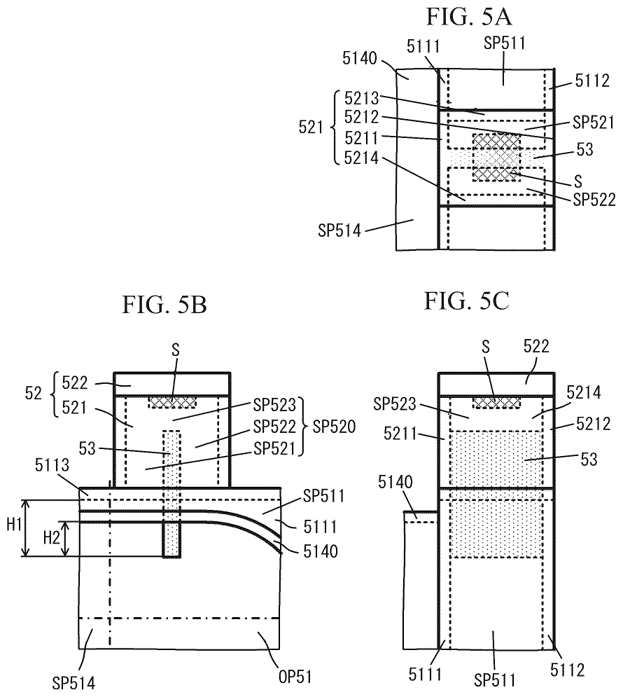

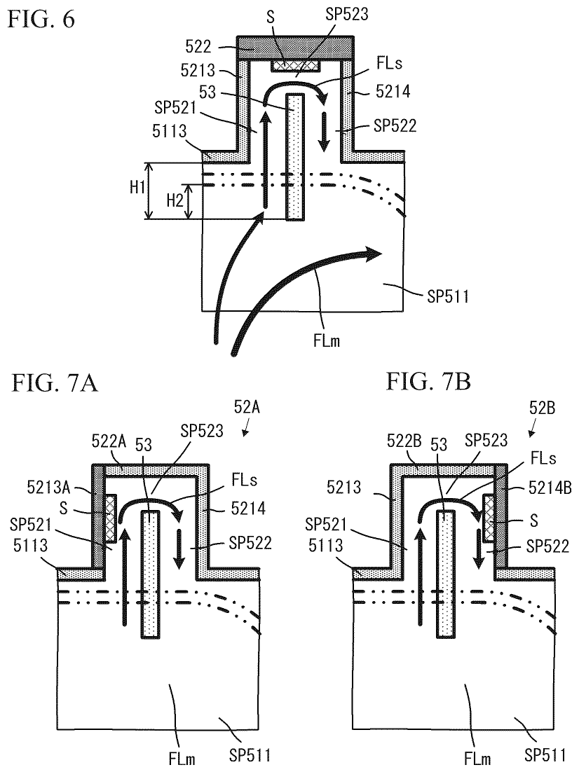

FIG. 5A is an enlarged plan view of a portion of a box according to an embodiment of the present disclosure, FIG. 5B is an enlarged first side view of a portion of a box according to an embodiment of the present disclosure, and FIG. 5C is an enlarged second side view of a portion of a box according to an embodiment of the present disclosure. FIG. 6 is a further enlarged side cross-sectional view of a portion of a box according to an embodiment of the present disclosure. FIG. 6 is a view viewed in the same direction as FIG. 5C.

A box 52 includes a tube 521 and the circuit board 522. The tube 521 protrudes to the exterior of the air pipe 50 from the wall 5113 of the first tubular body 511. The box 52 corresponds to a protrusion in the present disclosure.

The tube 521 includes a wall 5211, a wall 5212, a wall 5213, and a wall 5214. The wall 5211, the wall 5212, the wall 5213, the wall 5214, and the circuit board 522 correspond to a protrusion defining wall of the present disclosure. The wall 5211 and the wall 5212 face each other, and the wall 5213 and the wall 5214 face each other. The wall 5211 and the wall 5212 are connected to the wall 5213 and the wall 5214. The wall 5211 is parallel to the wall 5111 of the first tubular body 511, and connected to the wall 5111. The wall 5212 is parallel to the wall 5112 of the first tubular body 511, and connected to the wall 5112. The wall 5113 and the wall 5114 are perpendicular to the wall 5111 and the wall 5112 of the first tubular body 511. The wall 5113 is disposed closer to the wall 5115 of the first tubular body 511 than the wall 5114.

The tube 521 has a first opening connected to the first internal space SP511 of the first tubular body 511. The tube 521 has a second opening closed by the circuit board 522. Specifically, the box 52 is defined by the tube 521 and part of the circuit board 522, and has an internal space SP520 enclosed by the tube 521 and the circuit board 522. The sensor S is mounted on the surface of the circuit board 522 facing the internal space SP520.

The wall 53 is disposed in the internal space SP520 (a secondary space) of the box 52. More specifically, the wall 53 is a flat board. The wall 53 is disposed while having the flat board surfaces parallel to the wall 5213 and the wall 5214 of the tube 521.

The first end of the wall 53 in the width direction is connected to the wall 5211. The second end of the wall 53 in the width direction is connected to the wall 5212. The width direction of the wall 53 is thus parallel to the wall 5213 and the wall 5214, perpendicular to the direction in which the first tubular body 511 extends (the longitudinal direction), and perpendicular to the direction in which the wall 53 described below protrudes into the first internal space SP511.

A first end (an upper end) of the wall 53 in the height direction is disposed in the internal space SP520, at a position away from the circuit board 522. In this structure, the internal space SP520 includes a space SP521 (a first secondary space) between the wall 53 and the wall 5213, a space SP522 (a second secondary space) between the wall 53 and the wall 5214, and a space SP523 (a third secondary space) between the wall 53 and the circuit board 522. The space SP521 and the space SP522 are connected to each other through the space SP523.

The wall 53 protrudes into the first internal space SP511 of the first tubular body 511 by a height H1 at a second end (a lower end) in the height direction. When viewed in a direction perpendicular to the inlet port OP51 (in a front view), the wall 53 overlaps an area of the inlet port OP51, and protrudes into the inlet port OP51 by a height H2 at the second end (a lower end) in the height direction.

### Air Current in Air Pipe 50

As indicated with bold arrows in FIG. 3 and FIG. 6, in the air pipe 50, a main current FLm mainly used as a function of the CPAP machine 10 flows in from the inlet port OP51, passes through the fourth internal space SP514, the first internal space SP511, the third internal space SP513, and the second internal space SP512 in this order, and flows out from the outlet port OP52. This flow path through which the main current FLm flows corresponds to a main current path.

As illustrated in FIG. 6, at this time, the wall 53 protruding into the first internal space SP511 blocks part of the main current FLm, and causes a subsidiary current FLs. The subsidiary current FLs passes through the internal space SP520 of the box 52, and returns to the first internal space SP511. More specifically, the subsidiary current FLs flows through the space SP521, the space SP523, and the space SP522 in this order, and returns to the first internal space SP511. The portion defined by the first tubular body 511, the wall 5213 of the box 52, and the wall 53 and where the internal space SP521 and the first internal space SP511 are connected corresponds to a first communicating port of the present disclosure. The portion defined by the first tubular body 511, the wall 5214 of the box 52, and the wall 53 and where the internal space SP522 and the first internal space SP511 are connected corresponds to a second communicating port of the present disclosure.

Thus, the subsidiary current FLs flows near the circuit board 522 along the surface on which the sensor S is mounted. Thus, the sensor S is effectively cooled by the subsidiary current FLS.

The sensor S that is cooled in this manner can thus accurately measure the temperature or humidity of an air current. Particularly, in the CPAP machine 10, the blower 80 generates a large amount of heat, and thus the temperature of the circuit board 522 is more likely to rise. Thus, the sensor S is susceptible to the temperature rise of the circuit board 522. However, the CPAP machine 10 with the above structure can effectively cool the sensor S, and thus can accurately measure the temperature or humidity of an air current.

In the CPAP machine 10, the wall 53 protrudes into the first internal space SP511 of the first tubular body 511 by the height H1. Thus, the CPAP machine 10 can more reliably cause a subsidiary current FLs that flows into the box 52.

When the height H1 is appropriately set, the quantity of flow and the velocity of the flow of the subsidiary current FLs can be adjusted. Thus, the CPAP machine 10 can cause a subsidiary current FLs with a desired quantity of flow and a desired velocity of flow while appropriately controlling the effect on the main current FLm to cool the sensor S.

The wall 53 has flat plate surfaces perpendicular to the longitudinal direction of the first tubular body 511. Thus, the CPAP machine 10 can more efficiently cause the subsidiary current FLs that flows into the box 52.

The wall 53 has both ends in the width direction connected to the walls of the box 52. Thus, the entirety of the subsidiary current FLs that has flowed into the box 52 is guided to the sensor S. Thus, the CPAP machine 10 can efficiently supply the subsidiary current FLs to the sensor S, and can effectively cool the sensor S.

The wall 53 protrudes into the inlet port OP51 by the height H2. Thus, the CPAP machine 10 can more reliably allow a gas that has flowed in through the inlet port OP51 to flow into the internal space SP520 of the box 52. An adjustment of the height H2 can adjust the quantity of flow and the velocity of the flow of the subsidiary current FLs. Thus, the CPAP machine 10 can cause a subsidiary current FLs at a desired quantity of flow and a desired velocity of flow while appropriately controlling the effect on the main current FLm to cool the sensor S.

As described above, the wall 53 is disposed adjacent to the inlet port OP51. Specifically, the wall 53 is disposed at a position closer to the inlet port OP51 than to the outlet port OP52. The opening area of the inlet port OP51 is greater than the opening area of the outlet port OP52. Thus, in the air pipe 50, the air current flows slower at the inlet port OP51 than at the outlet port OP52. Thus, the subsidiary current FLs flows at a low velocity. The CPAP machine 10 can thus reduce the noise or pressure drop caused by the subsidiary current FLs.

Beyond the third tubular body 513 (the third internal space SP513), the gas flow path reduces the opening area (forms a narrow portion of an air pipe), and the gas flows at a higher velocity. The box 52 and the wall 53 disposed upstream from the third internal space SP513 have a greater opening area, and thus the gas flows at a lower velocity. Thus, the CPAP machine 10 can reduce the noise or pressure drop.

Due to the shapes of the second internal space SP512 and the third internal space SP513, a pressure difference occurs between the gas immediately after being sucked and the space accommodating the blower 80, and thus the quantity of the flow of the gas can be measured. The noise of the blower 80 is less easily let out of the housing 20 through the inlet port OP51. Thus, the CPAP machine 10 can reduce the noise let out of the housing 20.

The sensor S disposed closer to the inlet port OP51 can more directly measure the ambient gas. Thus, the CPAP machine 10 can accurately measure the temperature of the gas.

The internal space of the housing 20 accommodating the blower 80 and the air pipe 50 are divided by, for example, the wall 5112. Beyond the third internal space SP513, the gas flow path reduces the opening area and causes the pressure drop. Thus, a pressure difference occurs between the internal space of the housing 20 accommodating the blower 80 and the air pipe 50. As illustrated in FIG. 2A, the CPAP machine 10 includes a pressure difference sensor 60. The pressure difference sensor 60 measures the pressure difference between the internal space of the housing 20 accommodating the blower 80 and the air pipe 50. Thus, the CPAP machine 10 can determine the exhalation or inhalation or measure the quantity of flow. Although disposing the pressure difference sensor 60 forms a portion with a small opening area where the gas flows at a high velocity, the CPAP machine 10 can dispose the sensor S at an appropriate position by disposing the sensor S at the above position.

The wall 53 is disposed at a position in the first tubular body 511 near the connection end connected toward the second tubular body 512, in other words, at a position in the gas flow path near the outlet port OP52. Thus, the CPAP machine 10 can regulate the velocity of the flow of the subsidiary current FLs. Thus, the CPAP machine 10 can prevent the insufficient cooling of the sensor S.

The CPAP machine 10 flows the subsidiary current FLs into the box 52 with a substantially rectangular parallelepiped shape to cool the sensor S. Thus, the CPAP machine 10 can achieve a mechanism for cooling the sensor S with a simple structure. The CPAP machine 10 can achieve a small mechanism for cooling the sensor S. Thus, the CPAP machine 10 improves its design freedom.

The flat plate surfaces of the wall 53 are parallel to the wall 5213 and the wall 5214, but may be different from this. For example, the wall 53 may be parallel to the wall 5211 and the wall 5212, or the wall 53 may be parallel to a diagonal of the box 52 when the box 52 is viewed in a plan. Nevertheless, when the flat plate surfaces of the wall 53 are parallel to the wall 5213 and the wall 5214, the CPAP machine 10 can more efficiently cause a subsidiary current FLs.

In the above embodiment, the air pipe 50 in the CPAP machine 10 has a rectangular cross section (a cross section taken perpendicularly to the flow path), but may have a different cross section.

### Another Aspect of Arrangement of Sensor S

FIG. 7A and FIG. 7B are enlarged cross-sectional views of other examples of an arrangement of a sensor according to an embodiment of the present disclosure. As illustrated in FIG. 7A and FIG. 7B, an air pipe 50A and an air pipe 50B are different in terms of an arrangement of the sensor with respect to the air pipe 50. Other components of the air pipe 50A and the air pipe 50B are the same as those in the air pipe 50, and thus are not described.

In the example in FIG. 7A, a box 52A differs from the box 52 in that it includes a wall 522A and a circuit board 5213A. The circuit board 5213A is disposed parallel to the flat plate surfaces of the wall 53, and on a side of the first tubular body 511 opposite to the side that is connected to the third tubular body 513. The sensor S is disposed on the circuit board 5213A facing the wall 53 (in a space SP521).

In the example in FIG. 7B, a box 52B differs from the box 52 in that it includes a wall 522B and a circuit board 5214B. The circuit board 5214B is disposed parallel to the flat plate surfaces of the wall 53, and on a side of the first tubular body 511 connected to the third tubular body 513. The sensor S is disposed on the circuit board 5214B facing the wall 53 (in a space SP522).

With this structure, the CPAP machine can cool the sensor S.

### Another Aspect of Diverting Wall

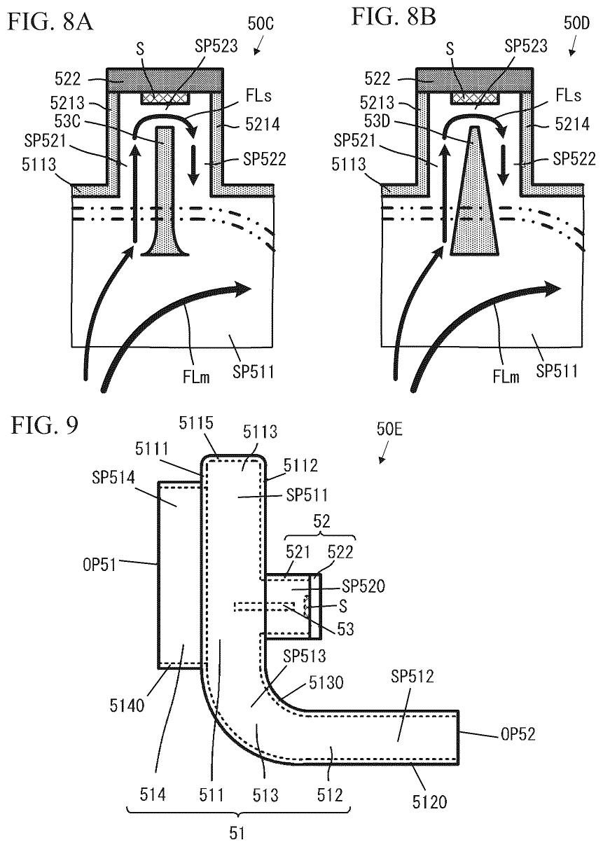

FIG. 8A and FIG. 8B are enlarged cross-sectional views of other examples of a diverting wall according to an embodiment of the present disclosure. As illustrated in FIG. 8A and FIG. 8B, an air pipe 50C and an air pipe 50D differ from the air pipe 50 in terms of the shape of the diverting wall. Other components of the air pipe 50C and the air pipe 50D are similar to those of the air pipe 50, and thus are not described.

In the example in FIG. 8A, a wall 53C has a uniform thickness substantially throughout, but increases the thickness toward its lower end, or a lower end portion protruding into the first internal space SP511 of the first tubular body 511.

In the example in FIG. 8B, a wall 53D gradually increases its thickness toward its lower end protruding into the first internal space SP511 of the first tubular body 511 from its upper end adjacent to the circuit board 522.

With these components, the CPAP machine can more reliably cause the subsidiary current FLs, and cool the sensor S.

### Another Example of Arrangement of Box

FIG. 9 is a diagram of another example of an arrangement of a box according to an embodiment of the present disclosure. As illustrated in FIG. 9, an air pipe 50E differs from the air pipe 50 in terms of an arrangement of the box 52. Other components of the air pipe 50E are the same as those of the air pipe 50, and thus are not described.

In the air pipe 50E, the box 52 is connected to the wall 5112. The flat plate surfaces of the wall 53 are perpendicular to the longitudinal direction of the first tubular body 511.

The CPAP machine including the air pipe 50E with this structure can cause the subsidiary current FLs, and cool the sensor S.

The box 52 may be connected to the wall 5114. Instead, the box 52 may be connected to the wall 5111. When the box 52 is connected to the wall 5111, the box 52 is connected to a portion of the first tubular body 511 closer to the third tubular body 513 than to a portion to which the fourth tubular body 514 is connected.

Alternatively, the box 52 may be connected to the third tubular body 513 or the second tubular body 512. Nevertheless, the box 52 is preferably connected to the first tubular body 511 because, as described above, the sensor S can be more effectively cooled by the subsidiary current FLs from the air current flowing further upstream in the air pipe.

### Cutouts Around Sensor

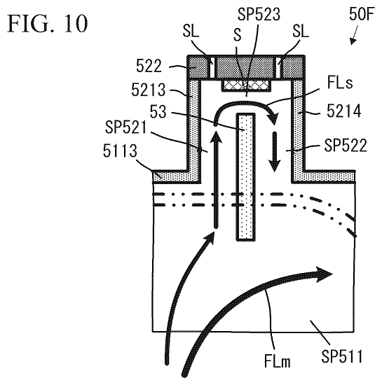

FIG. 10 is a diagram of another example of a box according to an embodiment of the present disclosure. As illustrated in FIG. 10, an air pipe 50F differs from the air pipe 50 in terms of the circuit board 522 defining the box 52. Other components of the air pipe 50F are the same as those of the air pipe 50, and thus are not described.

The circuit board 522 includes a groove SL around a portion where the sensor S is mounted. The groove SL extends through the circuit board 522 in the thickness direction. Instead, the groove SL does not have to extend through the circuit board 522 in the thickness direction as long as it has an appropriate depth. Nevertheless, the groove SL preferably extends through the circuit board 522 in the thickness direction.

With this structure, the sensor S is less susceptible to the heat from the outside of the groove SL of the circuit board 522. This structure can thus prevent the undesired temperature rises of the sensor S.

The width of the groove SL is determined based on the heat-blocking properties and the flow path resistance against the subsidiary current FLs. Specifically, preferably, the groove SL has high flow path resistance against the subsidiary current FLs. Thus, the air pipe 50F can reduce the subsidiary current FLs flowing into the groove SL.

1) A CPAP machine, comprising: a housing that includes a first opening and a second opening; an air pipe that is disposed in the housing, and has an inlet port continuous with the first opening, and an outlet port; a blower that is disposed in the housing, and guides a gas that has flowed in from the outlet port to the second opening; and a sensor that measures a temperature or a humidity of a gas that passes through an internal space of the air pipe, wherein the air pipe includes a protrusion that protrudes outward from a wall of the air pipe, and a diverting wall disposed in the protrusion, and wherein the diverting wall is at least partially located in the protrusion, defines a first communicating port and a second communicating port together with a protrusion defining wall defining the protrusion, and is disposed to allow the first communicating port and the second communicating port to be connected to each other in the protrusion, and the sensor is disposed at the protrusion defining wall.

2) The CPAP machine according to (1), wherein the diverting wall protrudes into a space located further inward than a wall of the air pipe defining a main current path.

3) The CPAP machine according to (1) or (2), wherein the diverting wall is connected to the protrusion defining wall at two ends perpendicular to a direction in which the protrusion protrudes.

4) The CPAP machine according to any one of (1) to (3),
wherein the diverting wall has a plate shape having a flat plate surface, and the flat plate surface of the diverting wall is perpendicular to a direction in which a portion of the air pipe at which the protrusion is connected extends.

5) The CPAP machine according to any one of (1) to (4), wherein the diverting wall is disposed in the air pipe at a position closer to the inlet port than to the outlet port.

6) The CPAP machine according to any one of (1) to (5), wherein the diverting wall overlaps the inlet port when the inlet port is viewed from a front.

7) The CPAP machine according to (6), wherein the diverting wall is disposed closer to the outlet port than a center when the inlet port is viewed from the front.

8) The CPAP machine according to any one of (1) to (7), wherein the protrusion defining wall defining the protrusion at which the sensor is disposed is a circuit board on which the sensor is mounted, and wherein the circuit board has a groove around the sensor.

9) The CPAP machine according to any one of (1) to (8), wherein the air pipe includes a narrow portion with an opening area smaller than an opening area of the inlet port, and wherein the diverting wall is disposed upstream from the narrow portion.

What is claimed is:

1. A CPAP machine, comprising:
a housing including a first opening and a second opening;
an air pipe disposed in the housing, and having an inlet port continuous with the first opening, and an outlet port;
a blower disposed in the housing, and configured to guide a gas flowed in from the outlet port to the second opening; and
a sensor configured to measure a temperature or a humidity of a gas passing through an internal space of the air pipe,
wherein the air pipe includes a protrusion protruding outward from a wall of the air pipe, and a diverting wall disposed in the protrusion, and
wherein the diverting wall is at least partially located in the protrusion, defines a first communicating port and a second communicating port together with a protrusion defining wall defining the protrusion, and is disposed to allow the first communicating port and the second communicating port to be connected to each other in the protrusion, and the sensor is disposed at the protrusion defining wall.

2. The CPAP machine according to claim 1,
wherein the diverting wall protrudes into a space located further inward than a wall of the air pipe defining a main current path.

3. The CPAP machine according to claim 1,
wherein the diverting wall is connected to the protrusion defining wall at two ends perpendicular to a protruding direction of the protrusion.

4. The CPAP machine according to claim 1,
wherein the diverting wall has a plate shape having a flat plate surface, and the flat plate surface of the diverting wall is perpendicular to an extending direction of a portion of the air pipe at which the protrusion is connected.

5. The CPAP machine according to claim 1,
wherein the diverting wall is disposed in the air pipe at a position closer to the inlet port than to the outlet port.

6. The CPAP machine according to claim 1,
wherein the diverting wall overlaps the inlet port when the inlet port is viewed from a front.

7. The CPAP machine according to claim 6,
wherein the diverting wall is disposed closer to the outlet port than a center when the inlet port is viewed from the front.

8. The CPAP machine according to claim 1,
wherein the protrusion defining wall defining the protrusion at which the sensor is disposed is a circuit board on which the sensor is mounted, and
wherein the circuit board has a groove around the sensor.

9. The CPAP machine according to claim 1,
wherein the air pipe includes a narrow portion with an opening area smaller than an opening area of the inlet port, and
wherein the diverting wall is disposed upstream from the narrow portion.

10. The CPAP machine according to claim 5,
wherein the diverting wall overlaps the inlet port when the inlet port is viewed from a front.
